(19) United States
(12) Patent Application Publication
(10) Pub. No.: US 2009/0081721 A1
(43) Pub. Date: Mar. 26, 2009

(54) HIGH-THROUGHPUT CELL ASSAYS

(75) Inventors: Matthew P. Meyer, Merced, CA (US); Miriam Barlow, Merced, CA (US); Shawn Newsam, Merced, CA (US)

Correspondence Address: FOLEY & LARDNER LLP, 975 PAGE MILL ROAD, PALO ALTO, CA, 94304, US

(73) Assignee: The Regents of the University of California

(21) Appl. No.: 12/101,104

(22) Filed: Apr. 10, 2008

Related U.S. Application Data
(60) Provisional application No. 60/911,206, filed on Apr. 11, 2007.
(60) Provisional application No. 61/018,870, filed on Jan. 3, 2008.

(51) Int. Cl. C12Q 1/04; G06F 17/00

(52) U.S. Cl. 435/34; 702/19

(57) ABSTRACT

This invention provides high-through put methods and systems to identify and/or classify cells present in a sample. In one aspect, the method identifies the cell by determining the amount of thermal energy required to disrupt cell membranes. In another aspect, a method is for determining if an agent such as a drug will inhibit the growth of a cell by monitoring the amount of energy required to maintain a substantially constant temperature in a sample containing the cell grown in the presence of an agent or drug is provided.

### CROSS REFERENCE TO RELATED APPLICATIONS

This application claims the benefit under 35 U.S.C. § 119(e) of U.S. Provisional Application Nos. 60/911,206, filed on Apr. 11, 2007 and 61/018,870, filed on Jan. 3, 2008, the contents of which are hereby incorporated by reference into the present disclosure.

### BACKGROUND OF THE INVENTION

Antibiotic resistance is a frequently encountered, expensive and often deadly threat to human health (1, 2). For example, in New York City in 1995, 1409 people died from methicillin resistant Staphylococcus aureus (MRSA) nosocomial (hospital acquired) infections. The monetary expense of those MRSA infections was estimated at $0.5 billion dollars (3, 4). The cost of treating hemodialysis patients infected by MRSA versus those infected by methicillin susceptible Staphylococcus aureus (MSSA) increased by more than 50% and patients with MRSA were 5.4 times more likely to die than those with MSSA (5). Nationally the monetary cost of antibiotic resistance for 1998 was estimated at $5 billion (4). Antimicrobial resistance has become such a common problem that empirical treatment of microbial infections is no longer an effective clinical strategy for numerous species types because of the emergence and spread of multiple drug resistant (MDR) strains of bacteria (6). Furthermore, efforts to reduce the occurrence of antimicrobial resistance by limiting or cycling antimicrobial consumption (7-9) have yielded inconsistent results (10, 11).

Currently available technology cannot significantly reduce the threat of antimicrobial resistance (7). However, this threat can, at the least, be monitored and contained by identifying and characterizing antimicrobial resistant bacteria, which in turn will reduce mortality rates associated with MDR infections (12). By increasing the ability of clinical microbiologists to rapidly deliver reliable strain identities and resistance profiles, the ability of physicians to appropriately treat infections will increase. The increased ability of physicians to appropriately prescribe antimicrobials will in turn improve patient outcomes. More rapid and reliable identification and characterization of infectious isolates may also enable physicians to prescribe narrow spectrum antimicrobials specific to the infection being treated rather than using broad-spectrum antimicrobials against an unknown infection. That change in prescription practice may in turn lower the occurrence of resistance to broad-spectrum antimicrobials. Improvement in the diagnostic capabilities of clinical microbiologists is likely to be a rapidly attainable improvement of clinical practices that will have great impact on improving our ability to effectively combat clinical antimicrobial resistance.

Currently available clinical techniques (13) to identify clinical isolates are lengthy, labor intensive, and in many cases, unreliable. Determining susceptibility phenotypes and species identities of infectious strains is a process that usually requires at least 72 hours. Current methods require two iterations of single colony isolation (overnight incubation required for each). Once clonal isolates are obtained, species identity is determined either manually or through automated approaches in which various metabolic, cell wall, and other informative characters are assessed. Manual identification is labor intensive and automated identification has a high consumables cost. Following identification, characterization of resistance phenotypes requires an additional overnight incubation to grow clinical isolates either in a panel of antimicrobials at multiple concentrations or on agar plates in which a concentration gradient of antimicrobials is established. The results of these tests are visually interpreted based on growth of the bacteria. Susceptibility testing is labor intensive and the span of time required for these tests can negatively impact patient outcomes. More rapid PCR (Polymerase Chain Reaction) based methods have been developed to determine whether specific resistance genes are present in a microbial sample. PCR does not however, assess the actual resistance phenotype of a microbe, which can range from complete susceptibility to complete insusceptibility because of differences in the expression of resistance genes.

As an example, antimicrobial susceptibility testing has been performed by Kirby-Bauer disk diffusion or minimum inhibitory concentrations. Disk diffusion testing is performed by coating an agar plate with a single strain of bacteria and applying a disk made of filter paper that contains a known quantity of antibiotic to the agar plate. The plate is then incubated overnight and as the bacteria grow, the antibiotic diffuses from the disk through the agar and kills the bacteria is regions where the concentration of the antibiotic exceeds the ability of the bacteria to inactivate, remove, or sequester the antibiotic. The death of the bacteria creates a zone of clearing around the disk and the diameter of that zone is measured, compared to clinical standards, and used to determine whether treatment with a specific antimicrobial is appropriate.

Minimum inhibitory concentrations are determined by inoculating several cultures of bacteria in separate tubes or wells of broth that typically contain a 2-fold serial dilution of an antimicrobial. Those cultures are then grown 18-20 hours and the lowest concentration of the antibiotic that completely inhibits growth is recorded and compared to clinical standards to determine if that antimicrobial is appropriate for use.

A major problem with both methods is that they require visible growth of the culture which takes several hours. Both methods are also labor intensive for data gathering because they require visual inspection and human judgment calls when the test yield unexpected results. Inconsistencies also exist between the results of the two methods. Isolates that are determined to be resistant to an antimicrobial by one testing method may be determined to be susceptible or intermediate by the other testing method.

Thus a need exists for a simple and reliable method for identifying the strains of microbial isolates. Similar shortcomings are attendant to the testing of other cell types, such as fungi or cancer cells, with their therapeutically relevant agents. Accordingly, more rapid and sensitive methods of determining drug resistance and susceptibility profiles of cells is needed. This invention satisfies this need and provides related advantages as well.

### SUMMARY OF THE INVENTION

This invention provides a method for identifying a cell, such as a microorganism, contained in a liquid sample by increasing the temperature of the liquid sample at a pre-determined constant rate and measuring the amount of power (energy as determined by power input) necessary to maintain that temperature at a substantially constant rate. This measured amount of power or energy optionally can be digitally or graphically recorded and then compared to the amount of power measured under substantially identical conditions for at least one reference cell sample or microorganism. If the measured amounts of power or energy is substantially identical between the unknown sample of cells or microorganism and the reference, then the cell or microorganism in the sample is the same as that of the reference cell or microorganism.

Also provided by Applicants is a system to perform this method, the system containing a processor and a computer-readable medium operably coupled to the processor, the computer-readable medium comprising instructions that, upon execution by the processor, perform operations comprising increasing the temperature of a liquid sample containing the microorganism at a pre-determined constant rate and measuring the amount of power (energy as determined by power input) necessary to maintain that temperature at a substantially constant rate. That information is then recorded, graphically or digitally, and compared to the measured amount of power or energy measured under substantially identical conditions for at least one reference sample or microorganism.

This invention also provides a method for determining if an agent affects the growth or metabolism of a cell such as a microorganism in a liquid sample by adding the agent to the sample containing the cell and increasing the temperature of the liquid sample at a pre-determined constant rate and measuring the amount of power or energy necessary to maintain that temperature at a substantially constant rate. The amount of power or energy is recorded digitally or graphically and then compared to a digital or graphical record of the amount of energy or power recorded for a sample of cell assayed under the same conditions, but without the presence of the agent. If the amount of energy or power is different between the two samples (the sample with agent and the sample without the agent) then the agent affects the growth of the cell and is a potential growth or metabolism inhibiting or promoting agent.

Also provided by Applicants is a system to perform this method, the system containing a processor and a computer-readable medium operably coupled to the processor, the computer-readable medium comprising instructions that, upon execution by the processor, perform operations described above. Prior to the comparison the information can be further analyzed or processed using methods described below and known in the art.

Yet further provided is a method for determining if an agent affects the growth or metabolism of a cell such as a microorganism contained in a liquid sample by measuring the energy required to maintain the temperature of the sample containing the agent at a substantially constant temperature and determining that the agent affects the growth or metabolism of the cell if the energy required to maintain the temperature of the sample is less than the measured energy of a reference sample that does not contain the agent. Also provided by this invention is a system to perform this method, the system containing a processor and a computer-readable medium operably coupled to the processor, the computer-readable medium comprising instructions that, upon execution by the processor, perform operations comprising measuring the amount of energy necessary to maintain the temperature of the sample at a substantially constant temperature, digitally or graphically recording this information and comparing it to the amount of power or energy recorded for a cell sample that does not contain the test agent. Prior to comparison, the information can be further analyzed using methods described below or known in the art. Also provided by this invention is a method for treating a patient in need thereof by performing the above method and administering to the patient the agent determined to inhibit or facilitate the growth of the cell or predetermined cell type. As is apparent to those skilled in the art, an effective amount of the agent is administered by any suitable means, intravenously, orally, intraperitoneally, in any suitable dose. Those can be empirically determined by the skilled artisan.

A method to identify agents that inhibit the growth of a cell such as a microorganism, comprising adding an effective amount of the agent to be tested to a suitable culture of cells and monitoring the energy produced by the culture as compared to a control culture of cells wherein no agent has been introduced, wherein the agent that reduces the energy produced by the cell culture as compared to control cell culture is identified as an agent that inhibits the growth of the cell. Also provided by this invention is a system to perform this method, the system containing a processor and a computer-readable medium operably coupled to the processor, the computer-readable medium comprising instructions to monitor the energy produced by the culture as compared to a control culture wherein no agent has been introduced, wherein the agent that reduces the energy produced by the culture of microorganism as compared to control culture is an agent that inhibits the growth of the microorganism. In one aspect, the energy is graphically or digitally recorded prior to the comparison. In a further aspect, the information is further analyzed prior to the comparison, using methods described below or known in the art.

In one aspect, the method provides a method comprising isothermal titrative calorimetry (ITC) to provide a rapid assessment of the effect of a test agent on the thermal output or metabolism of a cell. For example, the method is used to determine susceptibility of cells such as microorganisms to various antimicrobials more rapidly than current susceptibility testing methods. The method is accomplished by measuring differences in heat output from growing cultures of cells that are either exposed or not exposed to a particular compound or other agent. In sum and as described in more detail herein, the inventions have broad applicability for the determination of the effect, both inhibitory or stimulatory, of any test substance on the thermal output or metabolism of a cell of interest.

### BRIEF DESCRIPTION OF THE FIGURES

FIG. 1 depicts a block diagram of a calorimetry system in accordance with an exemplary embodiment.

FIG. 2 depicts a flow diagram illustrating exemplary operations performed by the system of FIG. 1 in accordance with an exemplary embodiment.

FIG. 3, panels A to D, show representative DSC thermograms of E. coli. From 0° C. to 60° C., the thermogram characteristics are similar, but from 60° C. to 130° C. the thermograms are variable. Similarity in the thermograms in the range of 0° C. to 60° C. is genus specific while similarities in the temperature range of 60° C. to 130° C. as seen in FIGS. 3A and 3D is therefore a likely indicator of strain type and probably represents clones of the same strain.

FIG. 4, panels A and B, show representative DSC thermograms of K. pneumoniae (panel A) and K. oxytoca (panel B). The Klebsiella thermograms shown in FIGS. 4A and 4B show genus similarities, but also differences that may be species specific in the 0° C. to 60° C. range.

FIG. 5 shows data extracted from DSC-generated thermograms of different classes of bacteria projected onto the first two dimensions of the eigen-gram subspace. Nineteen (19) samples from six different bacteria classes were analyzed and compared: Acinetobacter represented as circles, E. coli represented as x's, Enterobacter represented as pluses, Klebsiella represented as asterisks, Proteus represented as squares, and Pseudomonas represented as diamonds. This figure shows that the bacteria classes are separated even in this two-dimension space.

FIG. 6 depicts ITC-generated thermograms of E. coli. 1×04 wild-type, antibiotic susceptible E. coli were incubated in the ITC chamber for 14,400 sec (4 hours) in 1 ml of Mueller-Hinton broth. H2O, ampicillin, or ciprofloxacin was injected into the chamber at 7,200 sec (2 hours). Exponential increase in energy was detected for each sample prior to injection, but after injection an exponential increase in energy only continued in the sample injected with H2O.

FIG. 7 shows ITC-generated thermograms of K. pneumoniae. 105 ampicillin resistant (MIC>1024 μg/ml) ciprofloxacin susceptible (MIC=0.125 μg/ml) K. pneumoniae were incubated in the ITC chamber for 14,400 sec (4 hours) in 1 ml of Mueller-Hinton broth. H2O, ampicillin, or ciprofloxacin was injected into the chamber at 7,200 sec (2 hours). Exponential increase in power (μW) was detected for each sample prior to injection, after injection an exponential increase in power continued in the sample injected with H2O and ampicillin but not in the sample injected with ciprofloxacin.

FIG. 8 depicts thermograms of P. mirabilis. 105 ampicillin resistant (MIC>1024 μg/ml) weakly ciprofloxacin resistant (MIC=4 μg/ml) P. mirabilis were incubated in the ITC chamber for 14,400 sec (4 hours) in 1 ml of Mueller-Hinton broth. H2O, ampicillin, or ciprofloxacin was injected into the chamber at 7,200 sec (2 hours). Exponential increase in power (μW) was detected for each sample prior to injection, after injection an exponential increase in power continued in all three samples continued after injection, though the rate for ciprofloxacin was lower than for ampicillin or H2O.

FIG. 9 depicts thermograms of A. baumanii. 105 wild-type, ampicillin resistant (MIC>1024), ciprofloxacin resistant (MIC>32 μg/ml) A. baumanii were incubated in the ITC chamber for 14,400 sec (4 hours) in 1 ml of Mueller-Hinton broth. H2O, ampicillin or ciprofloxacin was injected into the chamber at 7,200 sec (2 hours). Exponential increase in power (μW) was detected for each sample prior to injection, after injection an exponential increase in power continued in all three samples continued after injection.

### MODES FOR CARRYING OUT THE INVENTION

Throughout this disclosure, various publications, patents and published patent specifications are referenced by an identifying citation. Also within this disclosure are Arabic numerals referring to referenced citations, the full bibliographic details of which are provided immediately preceding the claims. The disclosures of these publications, patents and published patent specifications are hereby incorporated by reference into the present disclosure to more fully describe the state of the art to which this invention pertains.

As used herein, certain terms have the following defined meanings.

### DEFINITIONS

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for that intended purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention.

Accordingly, it is intended that the methods and compositions can include additional steps and components (comprising) or alternatively include additional steps and compositions of no significance (consisting essentially of) or alternatively, intending only the stated methods steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “isolated” means separated from constituents, cellular and otherwise, in which the cell or other cellular component are normally associated with in nature. In addition, a “concentrated”, “separated” or “diluted” cell or culture of cells is distinguishable from its naturally occurring counterpart in that the concentration or number of molecules per volume is greater than “concentrated” or less than “separated” than that of its naturally occurring counterpart.

As used herein, the term “microorganism” intends a microscopic or sub-microscopic organism whose genetic material is surrounded by a nuclear membrane. Mitosis may or may not occur during replication. Examples of microorganisms include but are not limited to bacteria, fungi, archaea and protists.

Differential Scanning Calorimetry (DSC) is the quantitative detection of the heat energy that is lost or gained in a given process and has been applied to estimate the heat capacity for any process that can be modeled as a phase transition. The basic principle underlying DSC is that when the sample undergoes a physical transformation such as phase transitions, more (or less) heat will need to flow to it than the reference to maintain both at the same temperature. Whether the process, constituting structural changes accompanying alterations in cellular component structure, is endothermic or exothermic determines the quantity of heat that must flow to the sample chamber (10, 26-28). Changes in heat flow are registered as features (peaks and valleys) in the thermogram. These features constitute a unique description of the microbial composition of the sample.

The result of a DSC experiment is a heating or cooling curve. This curve has been used to calculate enthalpies of transitions by integrating the peak corresponding to a given transition. It also can be shown that the enthalpy of transition can be expressed using the following equation:

ΔH=KA

where ΔH is the enthalpy of transition, K is the calorimetric constant, and A is the area under the curve. The calorimetric constant will vary from instrument to instrument, and can be determined by analyzing a well-characterized sample with known enthalpies of transition (27).

Isothermal Titrative Calorimetry (ITC) is a quantitative technique that directly measures the binding affinity, enthalpy changes and binding stoichiometry between two or more molecules in solution. Energy and entropy changes from these measurements can be determined. In the context of the present invention, such an interaction can be between molecules or molecules and cells.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Pluripotent embryonic stem cells can be distinguished from other types of cells by the use of marker including, but not limited to, October-4, alkaline phosphatase, CD30, TDGF-1, GCTM-2, Genesis, Germ cell nuclear factor, SSEA1, SSEA3, and SSEA4. A clone is a line of cells that is genetically identical to the originating cell; in this case, a stem cell.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

“Substantially homogeneous” describes a population of cells in which more than about 50%, or alternatively more than about 60%, or alternatively more than about 70%, or alternatively more than about 75%, or alternatively more than about 80%, or alternatively more than about 85%, or alternatively more than about 90%, or alternatively, more than about 95%, of the cells are of the same or similar species or phenotype, e.g. resistant to a certain antimicrobial agent such as antibiotics.

“Substantially heterogeneous” describes a cell population that is less than about 50% homogeneous.

“Affect or affects” means influences or to bring about a change in.

An “effective amount” is an amount sufficient to effect beneficial or desired results. An effective amount can be administered in one or more administrations, applications or dosages.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals and pets.

A “control” is an alternative subject or sample used in an experiment for comparison purpose. A control can be “positive” or “negative”. For example, where the purpose of the experiment is to determine the identity of a microorganism, it is generally preferable to use a control (a sample wherein the identity is known). A positive control can be an microorganism that is sensitive to a certain antibiotic and a negative control can be an microorganism that is resistant to a certain antibiotic.

“A measured thermal energy” intends that the energy transferred into or contacted with the liquid sample. The energy produced or required to maintain a physical state is referred to herein as “power,” and the terms may be used synonymously.

The term “thermal output” refers generally to the energy generated, both positive and negative, as a result of a biochemical or physical interaction. In the context of the present invention, such an interaction can be between molecules or molecules and cells. In the case of an interaction between a molecule and a cell, the thermal output can represent the aggregate or net effect of the molecule on the metabolism of the cell.

The term “metabolism” refers generally to the chemical and physical transformations in a cell responsible for cellular physiology and pathology in disease. Included within this definition are processes such as energy generation, the building of structural components, information transfer, the building and breakdown of cell organelles and cell walls, cell division and growth, cell death, among others, which constitute both normal cellular physiology and pathophysiology in disease.

The terms “susceptibility” or “sensitivity” used in the context of a cell and a compound, e.g., a therapeutic agent, refers generally to the ability of the compound to produce a physiological effect on the cell. Accordingly, for example, in the case of an antibiotic and a bacterial cell, the bacterial cell is sensitive or susceptible to the antibiotic if the antibiotic has a cytostatic or cytotoxic effect on the bacterial cell that prevents it from growing. Conversely, in the case of a growth factor and a cell, the cell is sensitive or susceptible to the growth factor, if contact between the growth factor and the cell results in the promotion of growth. In the context of the present invention, “susceptibility” or “sensitivity” can be measured by thermal output. Thus, a cell, e.g., a bacterial cell, is sensitive or susceptible to an antibiotic if the presence of the antibiotic reduces thermal out by 10, 20, 30, 40, 50, 60, 70, 80, 90, or 100%, and fractions in between, as compared to an untreated control, generally an exponentially growing culture.

The term “minimum inhibitory concentration” or “MIC” as used herein refers generally to the lowest concentration of a compound, e.g., an antibiotic, that will inhibit the growth of a cell, e.g., a bacteria, after a suitable incubation period. As used in the context of the present invention, this term refers to the concentration at which thermal output is substantially reduced to a point where addition of further compound does not result in a further reduction in thermal out put.

The terms “resistance” or “drug resistance” refers generally to the ability of a cell, e.g., a bacteria, to disable or prevent transport of an agent that would otherwise have an effect on that cell type, e.g., a cytostatic or cytotoxic effect in the case of an antibiotic.

### DETAILED DESCRIPTION OF THE INVENTION

This invention provides a method for identifying a cell contained in a sample comprising the steps of: a) increasing the temperature of the liquid sample at a pre-determined constant rate and measuring the amount of power necessary to maintain that temperature at a substantially constant rate; and b) comparing the amount of power measured for the liquid sample to the amount of power obtained from a reference sample, thereby identifying the cell as the same or different from the reference cell. In one aspect, the measured power is graphically or digitally recorded prior to the comparison and the graphical and/or digital representations of the data is compared. In a further aspect, the data if further analyzed prior to the comparison. The method can be practiced on prokaryotic or eukaryotic cells.

Applicants have discovered that the amount of energy necessary to disrupt cellular components for a cell such as a microorganism or other membrane-containing cell type is unique for each cell type and therefore can be used as an identifier of the cell or cell types within the sample. As such, any membrane containing cell, such as a prokaryotic or eukaryotic cell, can be identified by the methods of this invention. Such cells include, but are not limited to animal cells, plant cells, avian cells, fungi, yeast cells and microorganisms, such as bacteria. The methods of this invention can also be used to identify cells as they mature and thereby can be utilized to identify an undifferentiated stem cell from a more differentiated stem cell, for example. Thus, for convenience, when the term “microorganism” is referenced in this text in relation to Applicants' inventions, it should be understood although not always explicitly stated that any one of the above noted cells can be substituted into the inventions described herein.

Thus, in one aspect, the method is suitable for any microscopic or sub-microscopic organism whose genetic material is enclosed within a membrane, e.g., bacteria, fungi, archea and protists. The microorganism sample is not limited by its native environment and therefore any sample suspected of containing a microorganism would provide a suitable sample or any samples of cells will suffice. The method can be applied to microorganisms present in a clinical isolate, such as blood, urine, spinal fluid or other clinical samples as long as the sample allows for the transfer and measurement of thermal energy in the sample. Other samples are isolated from the industrial setting, such as a food source such as a fermentation broth that is typical in brewing and wine making. The sample may contain a substantially homogeneous population of the microorganism or it may be heterogeneous, i.e., containing more than one species, sub-species or genera. Any suitable method for obtaining the sample or microorganism is appropriate as long as interfering contamination is avoided to preserve the integrity of the data. For the purpose of illustration only, a small sample of fluid can be drawn or isolated from a patient under sterile conditions or a swab of the sample can be obtained from a surface or isolated from a patient under sterile conditions.

Prior to practice of the method, it may be desirable to culture or grow the sample under conditions that select for a certain cell type or microorganism that is suspected of being contained in the sample. For example, if a sample is isolated from a patient and one wishes to determine if the patient is infected with a certain drug-resistant bacteria, one can culture the sample under conditions that would select for the growth of that bacteria over others. Because thermal energy is applied to the sample using techniques that allow for measuring the change in the thermal energy of the sample over a period of time, if the sample is to be cultured prior to use in the claimed method, the culture conditions should not interfere with the transfer and measurement of energy in the sample.

In one aspect of this invention, the methods of this invention are carried out using DSC. Perkin-Elmer (Perkin-Elmer, DSC 2, see perkinelmer.com, last accessed on Dec. 28, 2007) sells a DSC which can be fitted with an Intracooler II to allow temperatures below 30° C. (10). Applicants have shown that DSC can be used to identify clinically relevant microorganisms such as bacteria because phenotypically distinct bacteria have cell components that differ in composition. DSC is a method that yields distinct peaks at temperatures where different cellular components lose structural integrity. Although others have used DSC to study thermotropic phase changes in membrane lipids, activation and germination of spores, the state of water in bacterial cells and thermal denaturation of whole cells and cell components (10 and references cited therein), Applicants believe they are the first to show that application of the principals of thermal denaturation and energy measurement can be used to identify an unknown cell type and therefore phenotype bacteria. The position of thermogram peaks provides a unique pattern that is indicative of the phenotype of the bacterial culture. Furthermore, the response of these peaks to external chemical perturbation holds promise for more distinctive characterization of microorganisms or other cell types using thermal energy.

In one aspect, the method of this invention is a method for phenotyping of taxonomically distinct microbes or cells using DSC. Approximately 10% of the intended analyte volume is composed of subcultured cells in growth media or a blood sample (for clinical isolates). This is added to the DSC chamber and allowed to grow to a density of about 10⁶ to about 10⁷ cells/mL as determined by heat output. The sample is then diluted in analyte buffer, e.g. salts or buffer with cross-linking additives such as carbodiimides, glutaraldehyde or membrane destabilizing ethylene diamine tetraacetate. The sample is then heated at the predetermined rate, e.g., about 1.0° C. per minute up through a variety of temperatures, e.g. up to about 50.0° C., about 60.0° C., about 70.0° C., about 80.0° C., about 90.0° C., about 100.0° C., about 110.0° C., about 120.0° C. about 130.0° C., about 140.0° C., about 150.0° C., about 160.0° C., about 170.0° C., about 180.0° C. about 190.0° C. or about 200.0° C. The resulting compensation in power required to maintain the temperature ramp is read as a thermogram. Features in the diagram are patterns that are taxonomically distinct. One purpose is to provide clinical identification of patient-specific bacterial pathogens. This information is crucial to both treatment and the maintenance of public health records.

Applicants have determined that if heat is applied to a cell sample in a controlled fashion, it disrupts cellular components over well-defined temperature ranges. The phase changes that accompany the disruption of these cellular components are measured as peaks in a DSC thermogram. In addition, information content is enriched by performing these analyses using different chemically treated buffers, such as those containing carbodiimides, glutaraldehyde or ethylene diamine tetraacetate.

Applicants also have found that when DSC is used to apply and measure thermal energy, media components such as proteins or other large molecules can interfere with measurements. Therefore, in one aspect, the cells can be centrifuged and the cell pellet re-suspended in a suitable buffer such as phosphate buffered saline (PBS, pH 7.0) just prior to analysis.

In one aspect, the sample is cultured in liquid culture medium to a density of from about 10³ to about 10⁸, or alternatively, from about 10⁴ to about 10⁸, or yet further from about 10⁵ to about 10⁷, or alternatively from about 10⁶ to about 10⁷, all in cells per mL. Alternatively, the sample can be diluted to a cell density of about 10³ to about 10⁸, or alternatively, from about 10⁴ to about 10⁸, or yet further from about 10⁵ to about 10⁷, or alternatively from about 10⁶ to about 10⁷, all in cells per mL. The sample can be a substantially homogenous population of cells, a clonal population of cells or alternatively, a substantially heterogeneous population of cells.

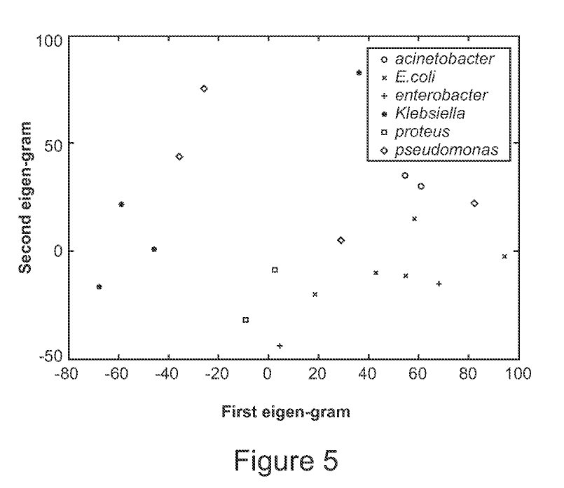

As used herein, the term “reference sample” intends one or more of a sample of cells, e.g., microorganisms, for which the change in thermal energy has been predetermined or the identify of which is known. Thus, in one aspect the reference sample is a control. For the purpose of illustration only, when the method is practiced to identify Pseudomonas from Staphylococcus, the reference stains can be one or both of each. In another aspect, when the method is practiced to identify drug resistant Pseudomonas aeruginosa from Pseudomonas fluourescens (a generally harmless relative used to produce antibiotics, protect plants and produce yogurt) the reference strain can be either or both of these. The results obtained from the test sample is then compared to the change in thermal energy of the control or reference strain. The energy can be digitally or graphically recorded and displayed prior to comparison, or yet further analyzed prior to comparison (see, e.g., FIG. 5).

After the sample is in condition for assaying, an effective amount of thermal energy is applied to disrupt cell membranes. A change in phase across a range of temperatures, e.g., from about 0.0° C. to about 150.0° C., or alternatively from about 0.0° C. to approximately 200.0° C., is preferred thus requiring heating of the sample at a rate at about 10° C. per minute for several hours to about 2.0° C. per minute, to about 3.0° C. to about 4.0° C. per minute, each for about 1.00 hour, or alternatively about 2.00 hour, or alternatively about 3.00 hour or alternatively about 4.00 hour.

The amount of energy necessary to maintain a substantially constant rate is recorded and compared to that simultaneously recorded or previously recorded for a reference sample. This is the thermogram of the specific sample. The pattern exhibited by the microorganism sample is then compared to the reference(s) and the reference having similar thermal properties is identified. In one aspect, the thermogram for energy applied below a temperature of 100.0° C. is utilized. In another aspect, the thermogram for energy applied a temperature below 90.0° C., or alternatively below 85.0° C., or alternatively below 80.0° C., or alternatively below 75.0° C., or alternatively below 70.0° C., or alternatively below 65.0° C. or yet further below 60.0° C. or yet further below 55.0° C., or yet further below 50.0° C. or yet further below 40.0° C. or yet further below 40.0° C. or yet further below 30.0° C. or yet further below 20.0° C., are compared to the reference sample. In a further aspect, the thermograms for a range of temperatures, e.g., between about 00° C. to about 60° C. or alternatively between about 60° C. and 130.0° C. are compared.

In one aspect, suitable positive and negative controls should be run simultaneously with the sample to confirm the integrity of the information obtained by the assay. In one aspect, the reference or control is de-gassed water (H₂O).

It is appreciated by those skilled in the art that the reference sample's thermogram need not be conducted at the same time as the test sample. A library of thermograms for each isolate or mixture of cells or yet further, the cells cultured in various culture mediums, can be obtained by conducting these tests and recording the information in, for example, a digital form as described herein, so that the sample can be compared to the information in the reference database. To that end, this invention also provides a method of preparing a thermogram or panel of thermograms for identifying a cell or microorganism of an unknown phenotype by applying and measuring by DSC to a microorganism or mixture of microorganisms of known phenotypes. The information across a range of temperatures and alternatively cultured under varying conditions, is stored in computer readable format (digital) or by graphical depiction of the thermal energy absorbed as a function of temperature.

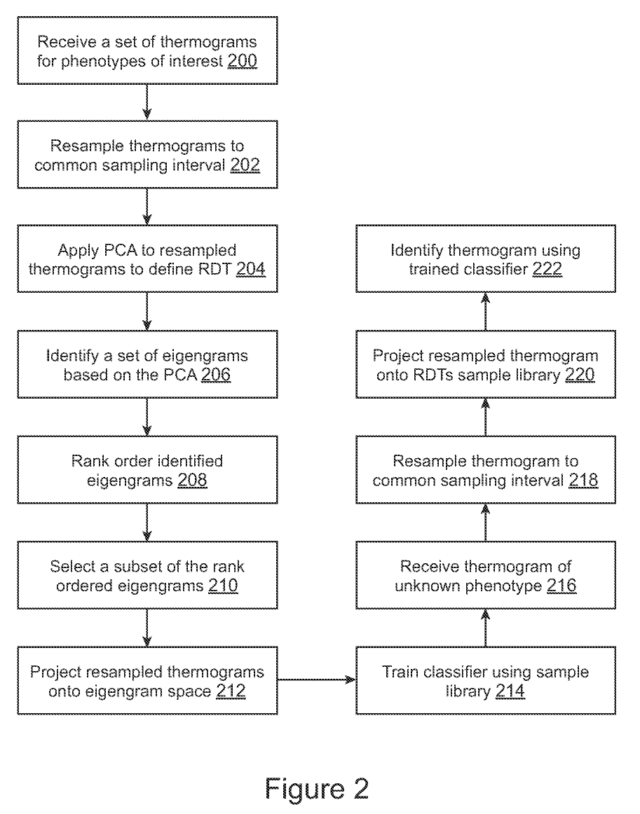

Substantial similarity of the thermogram to a reference thermogram identifies that the unknown cell type or microorganism is of the same species as the reference thermogram and is determined based on visual comparison or using a trained classifier (see FIG. 2). Accordingly, this invention also provides a library of thermograms for identifying an unknown organism likely to be present in certain environments, e.g. hospital settings, a brewery, in a vineyard or wine making establishment, sewage treatment plant, food packaging plants and high traffic areas like schools or airports. Application of the invention to cells such as stem cells can allow one to identify the identify of the cell, for example if the cell had differentiated or de-differentiated to a more or less mature phenotype.

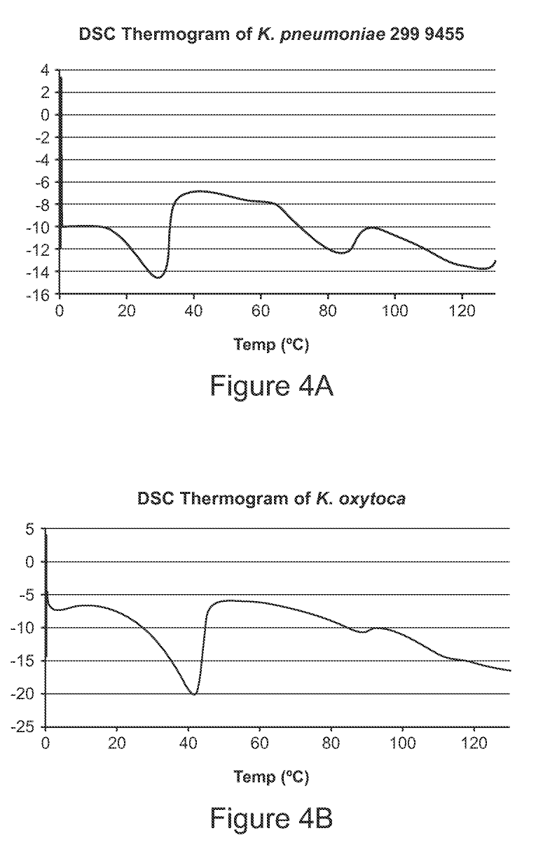

Further analysis of the data can be performed and is within the scope of this invention. The thermograms may be further analyzed prior to comparison with each other. One technique is the eigen-gram technique exemplified in FIG. 4 and described below is one method for comparing thermograms (or, equivalently, their graphical depictions). This can be done visually or using a trained classifier as described later in this application. Applicants have found that bilinear interpolation can be used to resample the thermograms of the references and the unknown isolates so that the data is aligned to common temperature intervals. For example, principal component analysis (PCA) has been used to identify rank-ordered set of eigen-gram subspace based on the eigen vectors and eigenvalues of the thermogram. Using this multivariant analytical tool, one can identify numerous species or phenotypes that are present in the same sample (see FIG. 4). Other suitable methods include, but are not limited to computing the mean square distance, computing and comparing discrete Fourier coefficients between thermograms, computing and comparing wavelet coefficients between thermograms and a Hilbert-Huang Transformation based comparison (28). As is apparent to those of skill in the art, it is unnecessary to repeat known samples each time an unknown sample is obtained for testing. Reference plots can be prepared from a variety of pre-selected cells or samples. It should be apparent to those of skilled in the art the various combinations of organisms may be selected from the environment is which they may be found. For example, related and unrelated species that are found in waste water may comprise a reference plot while those found in a hospital may comprise a separate reference plot. In addition, thermograms from certain disease resistant strains can be added to the multivariate analysis.

The above methods can be repeated and modified for multivariant and/or high-throughput analysis. The information or data can be obtained and expressed graphically or digitally in a computer-accessible storage medium.

This invention also is applicable with the use of an isothermal titration calorimeter (ITC). An ITC is composed of two identical cells made of a highly efficient thermal conducting material such as Hastelloy® alloy or gold, surrounded by an adiabatic jacket. Sensitive thermopile/thermocouple circuits are used to detect temperature differences between a reference cell and the sample cell. Prior to addition of a test substance, a constant power (<1 μW) is applied to the reference sample. This directs a feedback circuit, activating a heater located on the sample cell (VP-ITC users manual, MicroCal Inc, Northampton, Mass., USA. 2001). During the experiment, the test compound is titrated into the sample cell in precisely known aliquots, causing heat to be either taken up or evolved (depending on the nature of the reaction). Measurements consist of the time-dependent input of power required to maintain equal temperatures between the sample and reference cells.

In an exothermic reaction, the temperature in the sample cell increases upon addition of a test agent. This causes the feedback power to the sample cell to be decreased (as a reference power is applied to the reference cell) in order to maintain an equal temperature between the two cells. In an endothermic reaction, the opposite occurs; the feedback circuit increases the power in order to maintain a constant temperature.

Observations are plotted as the power in μcal/sec needed to maintain the reference and the sample cell at an identical temperature. This power is given as a function of time in seconds. As a result, the raw data for an experiment consists of a series of spikes of heat flow (power), with every spike corresponding to a ligand injection. These heat flow spikes/pulses are integrated with respect to time, giving the total heat effect per injection. The entire experiment generally takes place under computer control.

In embodiments of the present invention, the sample cell can contain cells in the presence of a test compound, while the reference cell will contain cells in the absence of the compound. With this experimental format, any of a number of types of determinations may be made. For example, the susceptibility or resistance of a variety of cells to the effects of therapeutic agents can be determined. Agents which, for example, have an inhibitory effect on cell growth or metabolism, would tend to decrease the production of heat from a cell after its addition to a cell as compared to a cell which was not exposed to the agent. If the cell is unaffected or resistant to the added agent, no change or a minimal change in heat production between the sample and reference cells would be observed. Conversely, agents which have a stimulatory effect on cells, such as increasing cell division or metabolism, would have the opposite effect, i.e., cells exposed to the agent would tend to show an increased production of heat as compared to cells which were not exposed to the agent.

As indicated below, the physiological consequences of the exposure of a cell to various agents, such as a bacterial cell to an antimicrobial or cancer cell to a chemotherapeutic or stem cell to a growth or differentiation factor, can be detected much earlier as a change in heat production as compared to a traditional visual assay relying on cell death or the lack of cell growth.

Thus, when one wishes to determine if a microorganisms is resistance or sensitivite to a test agent, one chamber will contain a bacterial sample in the absence of a test compound, while a second chamber will contain an identical bacterial sample to which an antimicrobial agent will be added.

In general, the susceptibility of bacterial strains can be tested by loading the chamber of an isothermal titrative calorimeter with a single bacterial strain, holding a constant temperature of 37° C. for a set time period (e.g., 14,400 seconds (4 hours)) and monitoring the power produced by the bacteria before and after application of an antimicrobial. Following the loading of the calorimeter, there is an initial energy spike within the first 1500 seconds (25 minutes) that is a normal equilibration period for a calorimeter. After the instrument and sample have equilibrated, the energy produced by a normally growing and dividing culture increases in an exponential manner, similar to a log phase growth curve of a microbial culture based on optical density (OD). Like the exponential increase in OD observed for a growing culture, the exponential increase in energy produced also seems to be associated with an increased number of cells in the medium because the number of cells retrieved from the calorimeter following a susceptibility test increases by about 2-3 orders of magnitude. (See Table 1).

For the purposes of minimum inhibitory concentration (MIC) determination of an antibiotic for a particular strain of bacteria, the invention can be used in a number of ways. In one mode of practice, sequential doses of an antimicrobial is applied at discrete intervals to a single culture until energy production is sufficiently inhibited to indicate that the minimum inhibitory concentration (MIC) of the antibiotic has been reached. The MIC value is used to characterize a culture as resistant or susceptible. One advantage of this mode of operation is that only a single chamber is required for susceptibility testing with each antimicrobial agent. In a second mode of operation, several discrete concentrations of antimicrobial agents are applied to several independent and identical cultures in separate calorimetry chambers.

Any of a variety of higher eukaryotic cells, e.g., mammalian cells, may also be used in the practice of the invention. Among such cells, cancer cells that may be used in the practice of the invention include, but are not limited to those derived from: Hodgkin's Disease, B-acute lymphoblastic lymphoma, prostate cancer, ovarian cancer, renal cancer, lung cancer, breast cancer, colon cancer, leukemia, multiple myeloma, hepatocarcinoma, Burkitt's lymphoma, and cervical carcinoma, among others. Stem cells may also find use in the practice of this invention. The cell types to be used in the practice of the invention may be supplied as purified cells, i.e., separated from other cell types, may be members of a heterogeneous population of cells, or may be part of a complex mixture of materials, such as a patient sample. When a purified cell population is used, methods known in the art for obtaining isolates of purified bacteria or fungi may be used, such as broth enrichment and isolation by plating to form single colonies. Methods for deriving clonal populations of mammalian cells, such as through use of serial dilution methods or FACS sorting, may also be used to obtain cells to be used in the practice of the invention.

Alternatively, patient samples may be used. Examples of the types of patient samples known in the art that may be used in the practice of the invention include: blood samples, urine samples and tissue biopsies.

In general, any type of compound may be tested using the methods of the invention for a measurable effect on heat production by a cell after contact with the compound. Accordingly, small molecules (e.g., antibiotics, chemotherapeutic agents, toxins), sugars, peptides, proteins (ligands, antibodies, enzymes, other biologics), and nucleic acids (siRNAs, antisense nucleic acids), among others, may be used in the practice of the invention depending on the cell type to be utilized.

Other uses of the invention include determining the effect of a chemotherapeutic agent on a cancer cell. Examples of chemotherapeutic agents that may be used in the practice of the invention include, but are not limited to: doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine methotrexate, fluorouracils, etoposide, taxol, taxol analogs, tamoxifen, fluorouracil, gemcitabine, and mitomycin.

Alternatively, a set of unknown compounds can be tested for their effect on a cell type of interest, e.g., a set of unknown compounds could be tested against a particular strain of pathogenic bacteria to identify new compounds that have antimicrobial properties. In such an embodiment, a library containing a large number of potential therapeutic compounds (e.g., a “combinatorial chemical library”) is screened using the ITC methods of the invention to identify compounds that have an effect on a cell, such as antimicrobial activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. Millions of chemical compounds can be synthesized through such combinatorial mixing of chemical building blocks (29).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

This invention also provides a method to detect physiological changes that precede cell death and therefore can be utilized to detect susceptibility to an antimicrobial agent. In one aspect, an agent can be added to the test sample and one can determine if the present of that agent affects the integrity of the cell membrane as measured by the change in thermal energy as a function of time and temperature. Thus, if the agent, e.g. a chemical compound (small molecule) or other biological affects the cell integrity as determined by the method, this agent can be administered to a patient infected or susceptible to infection with the microorganism. Because Applicants' method is quick, early identification of the specific strain infecting one or more patients is possible as well as identifying the agent or drug that is most effective to inhibit the growth of or kill the microorganism. This method is equally applicable to determine if an agent, such as a chemotherapeutic agent, affect cell metabolism or growth of a cell such as a cancer cell or stem cell.

Applicants also provide a method for determining if an agent affects the growth of a cell such as a microorganism contained in a liquid sample, comprising the steps of a) increasing the temperature of a first liquid sample at a pre-determined constant rate and measuring the amount of power necessary to maintain that temperature at a substantially constant rate; and b) adding the agent to a second sample of the cell at the same rate as the first pre-determined constant rate and measuring the amount of power necessary to maintain that temperature at a substantially constant rate; and c) comparing the amount of power measured for the first liquid sample to the amount of power measured for the second sample, thereby determining that the agent affects the growth of the cell if the measured energy of the sample is different than the measured energy of the second sample. In a further aspect, the results of the method or analysis are graphically or digitally recorded. In a further aspect, the cells are culture in different culture mediums and the resultant data is compared.

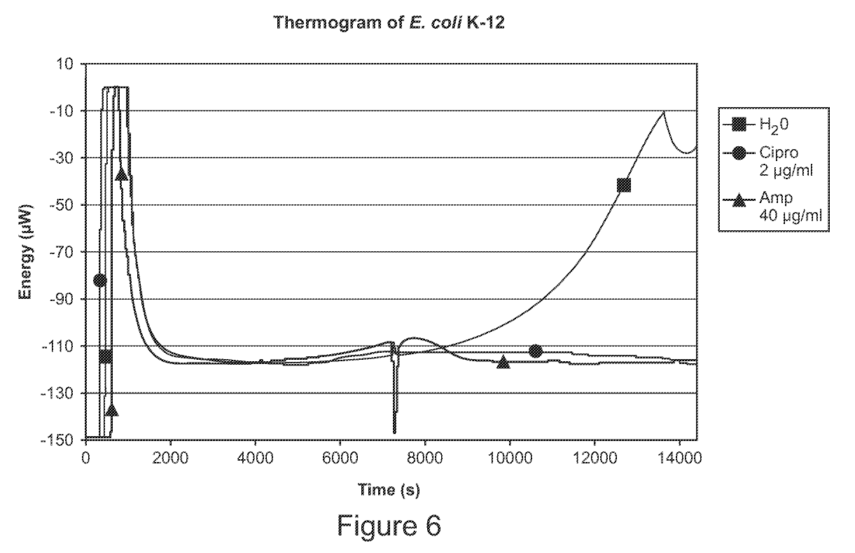

In one aspect, the method is practiced by loading into a Isothermal Titrative Calorimetry (ITC) various drugs or antibiotics to which the cells such as microorganisms may be susceptible. The antibiotics can be loaded at different rates and at different growth densities. The difference in power produced by the sample co-cultured with the antibiotics are then measured and compared to the same untreated culture or culture treated with water. The data is differentiated to find the maximum growth rate (dP/dt), and integrated to determine the total microjoules produced by the culture after an antimicrobial was injected. If the organism is resistant to the antibiotic, the maximum growth rate is similar to that when the co-cultured with water. If the microorganism is sensitive to the antibiotic, maximum dP/dt and total microjoule (μJ) for the antimicrobial treated sample are much lower than the untreated sample or the sample treated with water. See FIGS. 6 through 9 for exemplary ITC-generated growth data.

As is apparent to the skilled artisan, the phenotyping and antibiotic selection methods can be independently performed or performed in combination.

The samples can be any of the samples identified above and they also can be prepared using the methods described above. The energy monitored in this method distinguishes from Applicants' prior description in that the amount of energy necessary to maintain the temperature of the sample substantially (+/−1.0° C.) is measured in the presence and absence of a test agent such as a pharmaceutical, antibiotic or drug. In one aspect, the temperature is about 37° C. The temperature chosen for the assay will vary with the microorganism or cell being treated, its native environment or the environment of the host and can be determined by those of skill in the art.

Also similar to Applicants' prior method is that it can be modified for high throughput analysis and the results can be digitally stored. A library of references can be built from various modifications of the samples, the isolates, the culture conditions and the drugs or agents tested in the inventive methods.

The present methods can be further modified by applying sequential doses of an antimicrobial at discrete intervals to a single culture until energy production is sufficiently inhibited to indicate that the minimum inhibitory concentration (MIC) of the antibiotic has been reached. The MIC is the value used to categorize a culture as resistant or susceptible. The advantage of this method is that a single chamber is required for susceptibility testing with each antimicrobial. The disadvantage of this method is that it conceptually differs from currently accepted clinical susceptibility testing methods in which a culture of microbes is exposed to a single concentration of antimicrobials. This process could also be more time consuming when the MIC occurs at a particularly high concentration. In a yet further aspect, several discrete concentrations of antimicrobials are applied to several independent cultures in separate calorimetry chambers. This method is similar to current methods in which bacteria are cultured in discreet serial dilutions of antimicrobials. This method is also more rapid than sequential addition of an antimicrobial to a single chamber. The disadvantage of this method is that it reduces the throughput of the instrument because a single strain must occupy numerous calorimetry chambers for each antimicrobial. However, the instrument can be used flexibly to assay many strains simultaneously or in a mode where only a few strains are more rapidly assayed.

Also similar to the above methods, after a patient sample is collected and analyzed against a panel of possible drugs or agents, the patient suffering from the infection of the microorganism or alternatively, susceptible to infection, can be administered an effective amount of the drug, e.g., antibiotic, to patient to inhibit the growth or replication of the microorganism.

Further provided are computer systems for carrying out the methods described herein. In one aspect, a system is provided for identifying a microorganism, the system containing a processor; and a computer-readable medium operably coupled to the processor, the computer-readable medium comprising instructions that, upon execution by the processor, perform operations comprising identifying a microorganism contained in a liquid sample, comprising the steps of: a) increasing the temperature of the liquid sample at a pre-determined constant rate and measuring the amount of power necessary to maintain that temperature at a substantially constant rate; and b) comparing the amount of power measured for the liquid sample to the amount of power obtained from a reference sample, thereby identifying the microorganism as the same or different from the reference microorganisms.

Yet further provided is a system for determining if an agent affects, e.g., inhibits, the growth of a microorganism, the system containing a processor and a computer-readable medium operably coupled to the processor, the computer-readable medium comprising instructions that, upon execution by the processor, perform operations comprising determining if an agent affects the growth of a microorganism contained in a liquid sample, comprising the steps of: a) increasing the temperature of a first liquid sample at a pre-determined constant rate and measuring the amount of power necessary to maintain that temperature at a substantially constant rate; b) adding the agent to a second sample of the microorganism at the same rate as the first pre-determined constant rate and measuring the amount of power necessary to maintain that temperature at a substantially constant rate; and c) comparing the amount of power measured for the first liquid sample to the amount of power measured for the second sample, thereby determining that the agent affects the growth of the microorganism if the measured energy of the sample is different than the measured energy of the second sample. The methods and instructions can be further modified as described above.

Still further is provided a system for determining if an agent affects, e.g., inhibits the growth of a microorganism, the system containing a processor and a computer-readable medium operably coupled to the processor, the computer-readable medium comprising instructions that, upon execution by the processor, perform operations comprising measuring the amount of energy required to maintain the temperature of the thermal energy of the sample substantially constant as compared to a reference sample and identifying those agents that lower the amount of energy required to maintain the temperature of the sample substantially constant. The methods and instructions can be further modified as described above.

Further provided by this invention is a computer-readable medium comprising computer-readable instructions therein that, upon execution by a processor, cause the processor to identifying a classification of a microorganism, the instructions configured to cause a computing device to measure the change in the thermal energy of a biological sample as compared to a reference sample. An example of such a system is provided in Example 1, below.

Any of the above systems can be further modified by providing for testing the reference or test microorganism in the presence of an agent such as an antibiotic and determining if the agent inhibits the growth or kills the microorganism. These systems are particularly suited for identifying drugs that are specifically effective against treating microorganisms without the waste of time and resources inherent in current methodologies.

If an agent is identified as effective to inhibit the growth of a cell or microorganism, a patient in need of treatment can be administered an effective amount of the agent. Such therapeutic methods are further provided by this invention.

As shown herein, isothermal titrative calorimetry (ITC) is a more rapid method for susceptibility testing because it is based on thermal output from a growing culture rather than visible detection of the culture. It has been found that antibiotics have almost immediate effects on the thermal output of a growing culture. This enables a determination of the susceptibility of microbes in as little as 2.5 hours. At a minimum, ITC is likely to reduce the time required for diagnosing bacterial infections by 1 day. For blood-borne infections which typically consist of a single isolate and are also lethal in a short time frame, ITC may reduce the time to appropriate treatment of the infection to about 6 hours or less.

The use of isothermal titrative calorimetry as a method for determining the susceptibility of a bacterial strain to an antimicrobial is a better method than disk diffusion or minimum inhibitory concentration tests because it is more rapid (e.g., 2-4 hrs as opposed to 18-20) and because the output from an isothermal titrative calorimeter is entirely numeric, it can be automatically read and interpreted by computer software developed for that purpose. This method of susceptibility testing has greater potential for complete automation than the previously existing methods.

The following examples illustrate the concepts described herein.

### EXPERIMENTS

### Experiment No. 1

### Differential Scanning Calorimetry (DSC) System

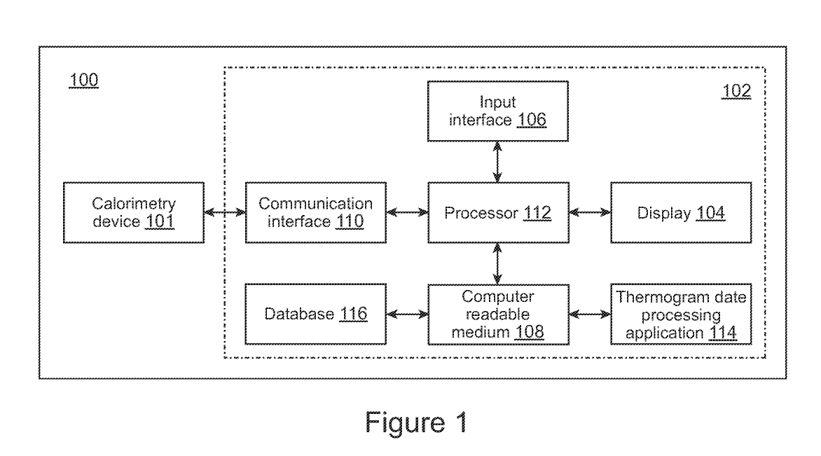

With reference to FIG. 1, a block diagram of a calorimetry system 100 is shown in accordance with an exemplary embodiment. Calorimetry system 100 may include a calorimetry device 101 and a computing device 102. Computing device 102 may include a display 104, an input interface 106, a computer-readable medium 108, a communication interface 110, a processor 112, a thermogram data processing application 114, and a database 116. In the embodiment illustrated in FIG. 1, calorimetry device 101 generates thermogram data. Computing device 102 may be a computer of any form factor. Different and additional components may be incorporated into computing device 102. Components of calorimetry system 100 may be positioned in a single location, a single facility, and/or may be remote from one another.

Display 104 presents information to a user of computing device 102 as known to those skilled in the art. For example, display 104 may be a thin film transistor display, a light emitting diode display, a liquid crystal display, or any of a variety of different displays known to those skilled in the art now or in the future.

Input interface 106 provides an interface for receiving information from the user for entry into computing device 102 as known to those skilled in the art. Input interface 106 may use various input technologies including, but not limited to, a keyboard, a pen and touch screen, a mouse, a track ball, a touch screen, a keypad, one or more buttons, etc. to allow the user to enter information into computing device 102 or to make selections presented in a user interface displayed on display 104. Input interface 106 may provide both an input and an output interface. For example, a touch screen both allows user input and presents output to the user.

Computer-readable medium 108 is an electronic holding place or storage for information so that the information can be accessed by processor 112 as known to those skilled in the art. Computer-readable medium 108 can include, but is not limited to, any type of random access memory (RAM), any type of read only memory (ROM), any type of flash memory, etc. such as magnetic storage devices (e.g., hard disk, floppy disk, magnetic strips, . . . ), optical disks (e.g., compact disk (CD), digital versatile disk (DVD), . . . ).

With reference to FIG. 2, exemplary operations associated with thermogram data processing application 114 of FIG. 1 are described. Additional, fewer, or different operations may be performed, depending on the embodiment. The order of presentation of the operations of FIG. 2 is not intended to be limiting. The functionality described may be implemented in a single executable or application or may be distributed among modules that differ in number and distribution of functionality from those described herein. A sample library of thermograms for a plurality of known phenotypes may be obtained and stored in database 116. In creating the library of thermograms for known phenotypes, sample thermograms are acquired from calorimetry device 101 for the phenotypes of interest. In an operation 200, a set of thermograms is received. For example, a set of thermograms for phenotypes of interest may be selected from the sample library of thermograms for input to thermogram data processing application 114 which receives the set of thermograms as an input. As another alternative, the set of thermograms may be streamed to computing device 102 from calorimetry device 101 as the calorimetry data is generated by calorimetry device 101 for a composition under study.

To correct for different temperature sampling intervals, the received thermograms are resampled to common (uniform) temperature intervals in an operation 202. This can be accomplished using any of a variety of resampling techniques such as bilinear interpolation. A reduced dimensional representation of the thermogram library is developed to support the classification of thermograms of unknown phenotypes. In an operation 204, principle component analysis (PCA) is applied to the resampled thermograms. As known to those skilled in the art, PCA is a technique used to reduce multidimensional data sets to lower dimensions for analysis. In an exemplary embodiment, the covariance method may be used to perform the PCA. As part of the PCA process, an Eigen value decomposition or singular value decomposition of the resampled thermograms is calculated.

In an operation 206, a set of Eigen grams (eigenvectors) is identified based on the PCA process. In an operation 208, the identified Eigen grams are rank-ordered based on their importance as determined from the singular values of the PCA decomposition. In an operation 210, a subset of the rank ordered Eigen grams is selected to represent the sample library by a reduced dimension thermogram (RAT). For example, a number of the rank ordered Eigen grams may be selected based on their Eigen value. The number of Eigen grams selected for the representation depends on the sample library. In general, the number of Eigen grams selected may range from five to 20 with the Eigen grams have the highest Eigen values being selected. The number of the rank ordered Eigen grams selected may be predetermined. In another exemplary embodiment, the number selected may depend on an evaluation of the trend in the Eigen values of the rank ordered Eigen grams. For example, the number of Eigen grams selected may be determined dynamically based on successive comparisons between adjacent eignevalues of the rank ordered eigengrams to identify when the successive comparisons indicate a sufficient drop in value to indicate that an adequate subset has been identified. For example, if the rank ordered eigenvalues are 100, 87, 79, 65, 0.2, 0.1, 0.01, four eigengrams may be selected. In another exemplary embodiment, the number of the rank ordered eigengrams selected may be by trial-and-error based on an observation of how a classification rate for a test dataset varies.

In an operation 212, the set of resampled thermograms are projected onto the lower-dimension eigengram space defined based on the rank ordered eigengrams to form RDT representations of the phenotypes. Thus, operations 200-212, may be used to define a library of thermograms, resampled thermograms, rank ordered eigengrams, and/or RDT representations of the phenotypes which form a reference database that may be stored in database 116.

In operation 214, a classifier is trained using the sample library. In an exemplary embodiment, a multi-class supervised classifier is trained. The classifier is used to identify the thermograms of unknown phenotypes. Any number of supervised classifiers can be used such as support vector machines, e.g., Bayes classifiers, linear classifiers, neural network classifiers, etc.

In an operation 216, a thermogram of an unknown phenotype is received for classification. Of course, as is readily understood by a person of skill in the art, the set of thermograms need not be obtained at the same time as the thermogram or using the same calorimetry device 101. In an operation 218, the received thermogram is resampled to the same temperature intervals used to create the sample library in operation 202. In an operation 220, an RDT is derived for the resampled thermogram by projecting it onto the lower-dimension eigengram space defined based on the rank ordered eigengrams which may be stored in database 116. In an operation 222, the trained classifier is used to identify the thermogram of the unknown phenotype.

In another exemplary embodiment, instead of using the trained classifier to perform the identification, the RDT of the unknown phenotype is plotted with the RDTs from the sample library. The identification of the unknown phenotype can be performed through visual inspection. Using this multivariant analytical tool, numerous species or phenotypes that are present in the same sample can be identified. As is apparent to a person of skill in the art, it is unnecessary to repeat known samples each time an unknown sample is obtained for testing. Reference plots can be prepared from a variety of pre-selected species. It should be apparent to a person of skill in the art that various combinations of organisms may be selected from the environment in which they may be found. For example, related and unrelated species that are found in waste water may comprise a reference plot while those found in a hospital may comprise a separate reference plot. In addition, thermograms from certain disease resistant strains can be added to the multivariate analysis.

The exemplary embodiments may be implemented as a method, apparatus, or article of manufacture using standard programming and/or engineering techniques to produce software, firmware, hardware, or any combination thereof to control a computer to implement the disclosed embodiments.

### Experiment No. 2

### Statistical Methods for Classifying Bacteria Based on their Thermograms

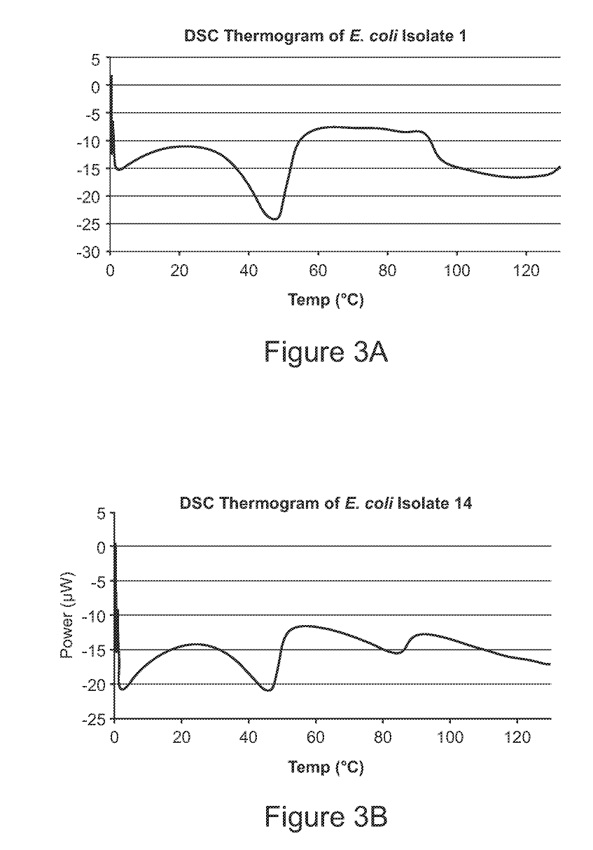

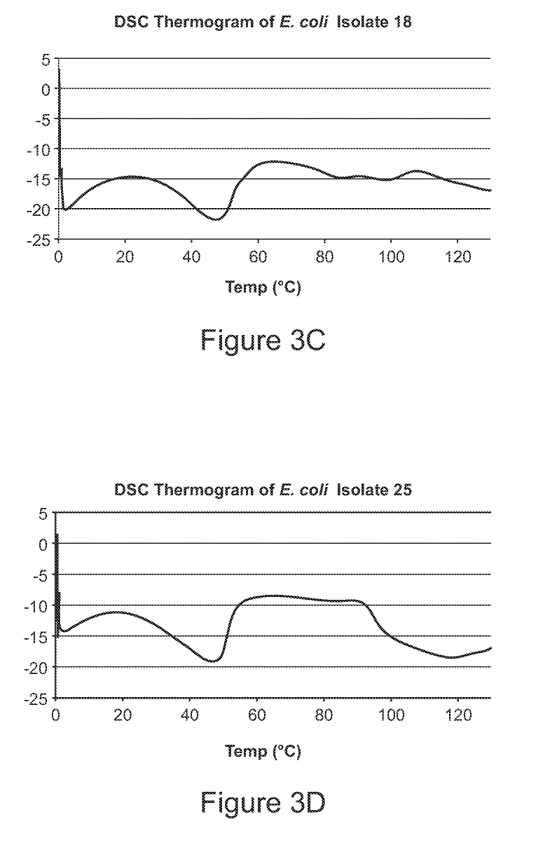

Differential scanning calorimetry (DSC) was performed on microbial samples that were at a concentration of 106 colony forming units (CFUs)/ml. Bacterial cultures were diluted in an isotonic buffer of inorganic salts and metal ions, loaded into the DSC chamber and then heated at a rate of 1.0° C./min from 00° C. to 130.0° C. As the sample was heated, the power difference between the sample chamber and the reference chamber was recorded as a thermogram. the thermogram features obtained from 0.0° C. to 60.0° C. contained genus specific features, but that the features present beyond that point were much more variable within genera. The consistency of features in the range of 0.0° C. to 60.0° C. suggest that DSC can be used to determine microbial isolate identity. The heterogeneity observed from 60.0° C. to 130.0° C. means that DSC could be used to identify specific strains and rapidly identify clonal outbreaks of resistant microbes in health-care settings. FIG. 3, panels A through D show representative E. coli thermograms.

### Experiment No. 3

### Statistical Methods for Classifying Bacteria Based on their Thermograms

Rapid interpretation of the thermograms to identify species is an essential component of high-speed analysis of DSC data. Since the energies for thermograms are sampled at different temperature values, bilinear interpolation is used to ‘resample’ the thermograms so that the data are aligned to common temperature intervals. By viewing these aligned signals as vectors indexed by temperature, the thermograms can be considered as points in a high-dimensional space. The dimensionality must be reduced before classifiers can be built that reveal clustering of classes of bacteria. Fortunately, many dimensionality reduction techniques are available for data present in high-dimensional space.

Principal component analysis (PCA) was used to identify a rank-ordered set of eigen-grams based on the eigenvectors and eigenvalues of the thermogram covariance matrix. The thermograms were then projected onto the lower-dimension eigen-gram subspace. The data reported herein demonstrate that the thermograms for different bacteria classes are separated in the eigen-gram subspace. FIG. 4 shows the projections onto the first two eigen-gram dimensions of thermograms for 19 samples from six different bacteria classes. This figure shows that the bacteria classes are separated even in this two-dimensional space. Note the class separation even in this low-dimension space. Nineteen (19) samples from six different bacteria classes are shown. It should be noted that the outlying Klebsiella isolate is K. oxytoca whereas the others are K. pneumoniae.

### Experiment No. 4

### Detection of Antimicrobial Resistance by Isothermal Titrative Calorimetry

The susceptibility of bacterial strains was tested by loading the chamber of an Isothermal Titrative Calorimeter (Calorimetry Sciences Corporation (CSC) and others are commercially available, see microcal.com/index, last accessed on Dec. 28, 2007) with a single bacterial strain, holding a constant temperature of 37° C. for 14,400 seconds (4 hours) and monitoring the power produced by the bacteria before and after application of an antimicrobial. Following the loading of the calorimeter, there is an initial energy spike within the first 1500 seconds (25 minutes) that is a normal equilibration period for a calorimeter. After the instrument and sample have equilibrated, the energy produced by a normally growing and dividing culture increases in an exponential manner, similar to a log phase growth curve of a microbial culture based on optical density (OD). Like the exponential increase in OD observed for a growing culture, the exponential increase in energy produced also seems to be associated with an increased number of cells in the medium because the number of cells retrieved from the calorimeter following a susceptibility test increases by 2-3 orders of magnitude. (See Table 1). A second spike in heat transfer being produced/absorbed in the calorimeter is associated with injection of either water or an antimicrobial into the injection chamber. This spike can be attributed to the enthalpy associated with dilution of the injected solution into the growth medium. When water is injected at 7200 seconds, the injection spike is followed by an exponential increase in energy until a sharp decrease presumably caused by a limited oxygen supply. When an antimicrobial is applied, the energy produced by the culture decreases significantly unless the strain is resistant to the antimicrobial, in which case the energy produced by the culture continues to increase in an exponential fashion. If a strain is susceptible, a rise in power is not observed. If the strain is resistant to the agent or antimicrobial, the exponential rise in power is observed.

The relationship between the power output of the samples and colony forming units of bacteria E. coli, K. pneumoniae, A. baumanii, and P. mirabilis collected from the chamber after analysis was evaluated and summarized in Tables 1 and 2. FIGS. 6 through 9 show individual thermograms for these bacteria analyzed in an ITC after introduction of two antimicrobials.

FIG. 6 depicts the thermograms of E. coli. 1×10⁴ wild-type, antibiotic susceptible E. coli were incubated in the ITC chamber for 14,400 sec (4 hours) in 1 ml of Mueller-Hinton broth. H₂O, ampicillin, or ciprofloxacin was injected into the chamber at 7,200 sec (2 hours). Exponential increase in energy was detected for each sample prior to injection, but after injection an exponential increase in energy only continued in the sample injected with H₂O.

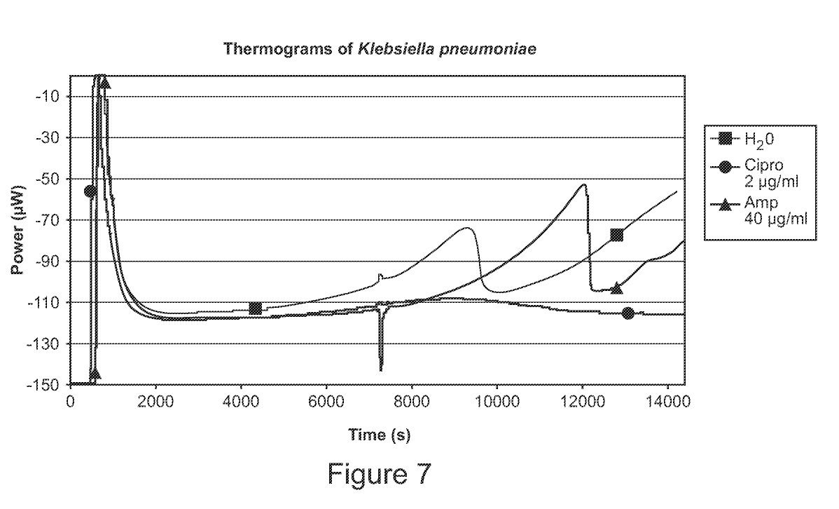

FIG. 7 shows thermograms of K. pneumoniae. 105 ampicillin resistant (MIC>10²⁴ μg/ml) ciprofloxacin susceptible (MIC=0.125 μg/ml) K. pneumoniae were incubated in the ITC chamber for 14,400 sec (4 hours) in 1 ml of Mueller-Hinton broth. H2O, ampicillin, or ciprofloxacin was injected into the chamber at 7,200 sec (2 hours). Exponential increase in power (μW) was detected for each sample prior to injection, after injection an exponential increase in power continued in the sample injected with H₂O and ampicillin but not in the sample injected with ciprofloxacin.

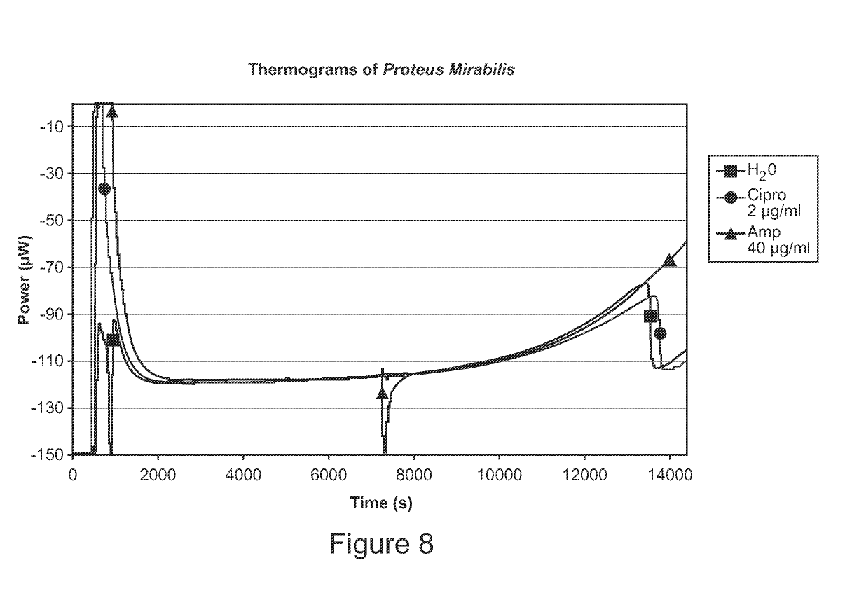

FIG. 8 depicts thermograms of P. mirabilis. 10⁵ ampicillin resistant (MIC>10²⁴ μg/ml) weakly ciprofloxacin resistant (MIC=4 μg/ml) P. mirabilis were incubated in the ITC chamber for 14,400 sec (4 hours) in 1 ml of Mueller-Hinton broth. H₂O, ampicillin, or ciprofloxacin was injected into the chamber at 7,200 sec (2 hours). Exponential increase in power (μW) was detected for each sample prior to injection, after injection an exponential increase in power continued in all three samples continued after injection, though the rate for ciprofloxacin was lower than for ampicillin or H₂O.

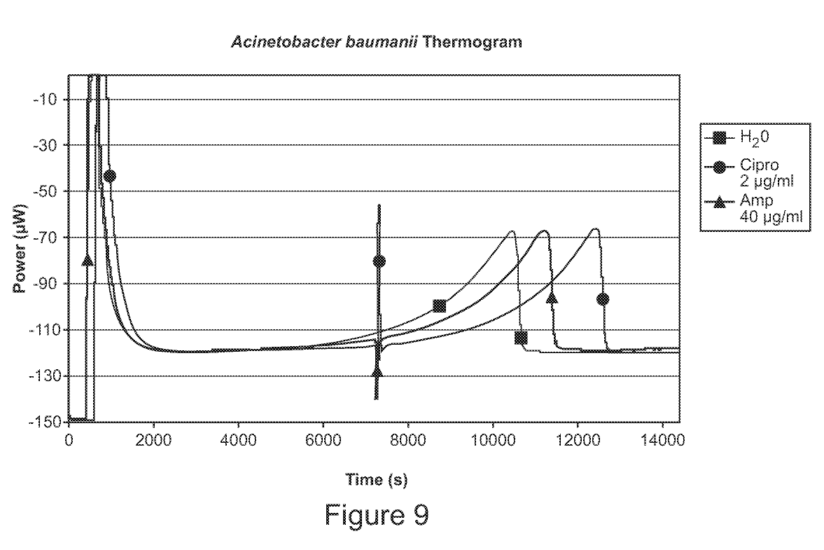

FIG. 9 depicts thermograms of A. baumanii. 10⁵ wild-type, ampicillin resistant (MIC
>10²⁴), ciprofloxacin resistant (MIC>32 μg/ml) A. baumanii were incubated in the ITC chamber for 14,400 sec (4 hours) in 1 ml of Mueller-Hinton broth. H₂O, ampicillin, or ciprofloxacin was injected into the chamber at 7,200 sec (2 hours). Exponential increase in power (μW) was detected for each sample prior to injection, after injection an exponential increase in power continued in all three samples continued after injection.

Unexpectedly, regardless of whether bacteria were treated with water or an antimicrobial to which they were susceptible, the number of colony forming units (CFUs) recovered from the chamber after the 4-hour monitoring period did not differ greatly (Table 1). While concentrations of antimicrobials injected into the ITC chamber which were sufficient to kill the bacteria after 16-20 hours, the duration of exposure to the antimicrobials was not sufficient to result in death during the final 2 hours of the ITC analysis. However, the affects of the antimicrobials on production of metabolic heat was still detectable. These affects correlate with resistance phenotypes determined by traditional susceptibility testing methods. These data indicate that the calorimeter detects physiological changes in the bacteria that are rapidly induced by antimicrobials, but that are not necessarily associated with death of the cells. The ability of ITC to detect physiological changes that precede death of the cells means that this is a method that can be used to detect susceptibility to an antimicrobial more rapidly than traditional assays, such as visual growth or cleaning of cultures.

TABLE 1
Number of microbes (CFUs) in calorimetry chamber

| | | After incubation with | | |
|---|---|---|---|---|
| | | | Ciprofloxacin | Ampicillin |
| | Loaded | H₂O | (2 μg/ml) | (40 μg/ml) |
| E. coli | 10⁴ | 1.9 × 10⁷ | 1.7 × 10⁷ | NA |
| K. pneumoniae | 10⁵ | 7.4 × 10⁷ | 3.6 × 10⁷ | 2.0 × 10⁷ |
| A. baumanii | 10⁵ | 1.6 × 10⁷ | 2.1 × 10⁷ | 3.0 × 10⁷ |
| P. mirabilis | 10⁵ | 7.2 × 10⁵ | 4.1 × 10⁶ | 9.0 × 10⁵ |

The difference in power produced by a culture treated with an antibiotic when compared to an equivalent culture treated with water is an indicator of the effectiveness of an antimicrobial against a specific bacterial strain. The data can be differentiated to find the maximum growth rate (dP/dt), and integrated to determine the total μJoules (μJ) produced by the culture after an antimicrobial was injected. Extraction of these data from the thermograms is provided in Table 2. When cultures are injected with an antimicrobial to which they are resistant, both the maximum dP/dt and the total μJ are similar to equivalent cultures that are injected with water. However, when the cultures are susceptible, max dP/dt and total μJ are much lower for cultures injected with antimicrobial than for cultures injected with water consistent and reproducible replicates were obtained.

TABLE 2
Maximum Increase and Total Energy Produced after Injection

| | | | |
|---|---|---|---|
| | | Ciprofloxacin | Ampicillin |
| Substance injected | H₂O | (2 μg/ml) | (40 μg/ml) |

Klebsiella pneumoniae (ciprofloxacin susceptible, ampicillin resistant)

| | | | |
|---|---|---|---|
| Max dP/dt (100 save) | 0.019 | 0.003 | 0.029 |
| Total μJ | 150,272 | 24,676 | 139,108 |

Proteus mirabilis (ciprofloxacin resistant, ampicillin resistant)

| | | | |
|---|---|---|---|
| Max dP/dt (100 save) | 0.016 | 0.012 | 0.027 |
| Total μJ | 88,014 | 79,439 | 161,844 |

Acinetobacter baumanii (ciprofloxacin resistant, ampicillin resistant)

| | | | |
|---|---|---|---|
| Max dP/dt (100 save) | 0.026 | 0.027 | 0.026 |
| Total μJ | 86,331 | 100,846 | 91,240 |

In addition to demonstrating that ITC can be used to detect antimicrobial susceptibility Applicants show that ITC can detect the degree of susceptibility of microbes to antibiotics. Bacteria were treated with different concentrations of ceftazidime (32 μg/ml, 128 μg/ml, 512 μg/ml) to a ceftazidime resistant strain of Proteus mirabilis (MIC 128 μg/ml) and measured the energy output of the treated cells. The differences in power output can be visualized in the thermograms (see FIGS. 6 through 9) and quantified in Table 2, demonstrate the ability of ITC to detect the inhibitory affects that antimicrobials have above the MIC threshold of an antimicrobial both visually and by the total μJoules produced by the culture after injection of the antimicrobial.

### EXPERIMENTAL DISCUSSION

Improved methods to rapidly identify and phenotypically characterize MDR strains of bacteria in clinical settings are likely to critically improve microbial treatment strategies. This invention describes the potential of two innovative approaches for rapid real time assessment of bacterial identity and susceptibility to antimicrobials. Differential Scanning Calorimetry (DSC) and Isothermal Titrative Calorimetry (ITC) are methods with the potential capability to rapidly identify bacteria and rapidly determine their antimicrobial susceptibility. Briefly, calorimetry is the quantitative detection of the heat energy that is lost or gained in a given process. DSC is a method by which one may estimate the heat capacity for any process that can be modeled as a phase transition. In bacteria there are a number of physical processes (denaturation or melting) that can be thought of as phase changes. They include denaturation of the ribosome, the cell wall, nucleic acids, and the cellular envelope as bacteria are heated (14-17). The application of DSC as a method for determining the identities of microbial isolates is novel and highly innovative because DSC has rarely been used on whole cells and has never been used to identify microbial organisms. ITC is a calorimetric method in which the temperature of the system is held constant and the energy required to maintain a constant temperature is quantified. ITC has frequently been used to measure the number of ligand receptors in a given sample based on the known concentration of ligand titrant. In this approach, clinically available antibiotics are used as ligands and whole cells as receptors. Metabolic heat produced by the bacteria is used to determine the effect of a known concentration of antimicrobials on growing microbial cultures. The application of ITC as a method to characterize the resistance phenotypes of microbes is highly innovative because ITC is rarely performed on whole cells (18) and is a fundamentally different approach for identification and characterization of infectious isolates than current approaches, which are based on PCR or visible bacterial growth.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

1. French, G. L. (2005) Adv Drug Deliv Rev 57, 1514-27.
2. McGowan, J. E., Jr. (2001) Emerg Infect Dis 7, 286-92.
3. Rubin, R. J., Harrington, C. A., Poon, A., Dietrich, K., Greene, J. A. & Moiduddin, A. (1999) Emerg Infect Dis 5, 9-17.
4. Harrison, P. F. & Lederberg, J. (1998) (National Academy Press, Washington, D.C.).
5. Reed, S. D., Friedman, J. Y., Engemann, J. J., Griffiths, R. I., Anstrom, K. J., Kaye, K. S., Stryjewski, M. E., Szczech, L. A., Reller, L. B., Corey, G. R., Schulman, K. A. & Fowler, V. G., Jr. (2005) Infect Control Hosp Epidemiol 26, 175-83.
6. Snyder, J. W., McDonald, L. C. & Van Enk, R. (2000) J Med Liban 48, 208-14.
7. Livermore, D. M. (2005) Lancet Infect Dis 5, 450-9.
8. Wise, R. (2004) J Antimicrob Chemother 54, 306-10.
9. McCaig, L. F., Besser, R. E. & Hughes, J. M. (2003) Emerg Infect Dis 9, 432-7.
10. Warren, D. K., Hill, H. A., Merz, L. R., Kollef, M. H., Hayden, M. K., Fraser, V. J. & Fridkin, S. K. (2004) Crit. Care Med 32, 2450-6.
11. Martinez, J. A., Nicolas, J. M., Marco, F., Horcajada, J. P., Garcia-Segarra, G., Trilla, A., Codina, C., Torres, A. & Mensa, J. (2006) Crit. Care Med 34, 329-36.
12. McGowan, J. E., Jr. & Tenover, F. C. (2004) Nat Rev Microbiol 2, 251-8.
13. National Committee for Clinical Laboratory Standards (2001) Performance standards for antimicrobial susceptibility testing. Supplemental tables. NCCLS Document M100-S11 (National Committee for Clinical Laboratory Standards, Wayne, Pa.).
14. Jackson, M. B. & Sturtevant, J. M. (1977) J Biol Chem 252, 4749-51.
15. Lee, J. & Kaletunc, G. (2002) Appl Environ Microbiol 68, 5379-86. Mackey, B. M., Miles, C. A., Parsons, S. E. & Seymour, D. A. (1991) J Gen Microbiol 137, 2361-74.
16. Mackey, B. M., Miles, C. A., Parsons, S. E. & Seymour, D. A. (1991) J Gen Microbiol 137, 2361-74.
17. Niven, G. W., Miles, C. A. & Mackey, B. M. (1999) Microbiology 145 (Pt 2), 419-25.
18. Aki, H., Nakashima, Y., Kawasaki, Y. & Niiya, T. (2006) Journal of Thermal Analysis and Calorimetry 85, 685-688.
19. Raymond, N. J., Blackmore, T. K., Humble, M. W. & Jones, M. R. (2006) Intern Med J 36, 765-72.
20. Russell, D. J. & Hansen, L. D. (2006) Thermochimica Acta 445, 151-159.
21. Barlow, M. & Hall, B. G. (2002) Antimicrob. Agents Chemother. 46, 1190-1198.
22. Barlow, M. & Hall, B. G. (2002) Genetics 160, 823-832.
23. Barlow, M. & Hall, B. G. (2003) Genetics 163, 1237-1241.
24. Welsh, K. J., Barlow, M., Tenover, F. & McGowan, J. E., Jr. (2004) Antimicrob Agents Chemother Salipante, S. J., Barlow, M. & Hall, B. G. (2003) Antimicrob Agents Chemother 47, 3840-5.
25. Dean, John A. The Analytical Chemistry Handbook. New York. McGraw Hill, Inc. 1995. pp. 15.1-15.5.
26. Pungor, Erno. A Practical Guide to Instrumental Analysis. Boca Raton, Fla. 1995. pp. 181-191.
27. Skoog, et al. Principles of Instrumental Analysis. Fifth Edition. New York. 1998. pp. 905-908.
28. Huang et al. (1998) Proc. R. Soc. Lond. A. 454:903-995.
29. Gallop et al. (1994) J. Med. Chem. 37(9):1233-1251.

What is claimed is:

1. A method for identifying a cell contained in a liquid sample, comprising the steps of:
a) increasing the temperature of the liquid sample at a pre-determined constant rate and measuring the amount of power necessary to maintain that temperature at a substantially constant rate; and
b) comparing the amount of power measured for the liquid sample to the amount of power obtained from a reference sample, thereby identifying the cell in the liquid sample as the same or different from the reference sample.

2. The method of claim 1, further comprising:
c) digitally recording the amount of power necessary to maintain the temperature at the substantially constant rate.

3. The method of claim 1, wherein the liquid sample is a culture medium.

4. The method of claim 1, wherein the cell is isolated from a patient sample.

5. The method of claim 1, wherein the cell is isolated from a food source.

6. The method of claim 1, wherein the cell is isolated from a feedstock sample.

7. The method of claim 1, wherein the liquid sample comprises a density of from about 10⁵ to about 10⁷ of the cell per mL of liquid sample.

8. The method of claim 1, wherein step a) is performed by a Differential Scanning Calorimeter (DSC) and the amount of power of step a) is expressed as a thermogram.

9. The method of claim 8, further comprising transforming the thermogram into a digital representation.

10. The method of claim 1, wherein step b) is performed by eigen-gram decomposition.

11. The method of claim 1, wherein the reference sample comprises a library of reference samples.

12. The method of claim 1, wherein step b) is performed by visual comparison.

13. The method of claim 1, wherein the sample comprises a substantially homogenous population of cells.

14. The method of claim 13, wherein the substantially homogenous population comprises a clonal population of cells.

15. The method of claim 1, wherein the sample comprises a substantially heterogeneous population of cells.

16. The method of claim 1, further comprising performing multivariate analysis of the amount of power measured from the reference sample.

17. The method of claim 1, wherein steps a) and b) are repeated with the same liquid sample.

18. The method of claim 1, further comprising digitally recording the amount of power measured in step a).

19. The method of claim 16, further comprising digitally storing the results of the multivariate analysis.

20. A method for determining if an agent affects the growth or metabolism of a cell contained in a liquid sample, comprising the steps of:
a) increasing the temperature of a first liquid sample containing the cell at a pre-determined constant rate and measuring the amount of power necessary to maintain that temperature at a substantially constant rate; and
b) adding the agent to a second sample containing the cell at the same rate as the first pre-determined constant rate and measuring the amount of power necessary to maintain that temperature at a substantially constant rate;
c) comparing the amount of power measured for the first liquid sample to the amount of power measured for the second sample, thereby determining that the agent affects the growth or metabolism of the cell if the measured power of the sample is different than the measured power of the second sample.

21. The method of claim 20, further comprising:
d) digitally recording the amount of power necessary to maintain the temperature at the substantially constant rate for the samples.

22. The method of claim 20, wherein the liquid sample is a culture medium.

23. The method of claim 20, wherein the cell is isolated from a patient sample.

24. The method of claim 20, wherein the cell is isolated from a food source.

25. The method of claim 20, wherein the cell is isolated from a feedstock sample.

26. The method of claim 20, wherein the first and second liquid sample comprises a density of from about 10⁵ to about 10⁷ of the cell per mL of liquid sample.

27. The method of claim 20, wherein the measuring steps of a) and b) are performed by a Differential Scanning Calorimeter (DSC) and the amount of power of steps a) and b) are expressed as a thermogram.

28. The method of claim 27, further comprising transforming the thermogram into a digital representation.

29. The method of claim 20, wherein step c) is performed by eigen-gram decomposition.

30. The method of claim 20, wherein step c) is performed by visual comparison.

31. The method of claim 20, wherein step c) is performed by digital comparison.

32. The method of claim 20, wherein the first and second sample comprises a substantially homogenous population of cells.

33. The method of claim 32, wherein the substantially homogenous population comprises a clonal population of cells.

34. The method of claim 20, wherein the first and second sample comprise a substantially heterogeneous population of cells.

35. The method of claim 20, further comprising:
d) performing multivariate analysis of the amount of energy measured for the first and second samples.

36. The method of claim 20, wherein all steps are repeated to the same sample or samples.

37. The method of claim 20, wherein the measuring of steps a) and b) are performed by visual analysis.

38. The method of claim 35, further comprising digitally storing the results of the multivariant analysis.

39. A method for determining if an agent affects the growth or metabolism of a cell contained in a liquid sample, comprising measuring the power required to maintain the temperature of the sample containing the agent at a substantially constant temperature and determining that the agent affects the growth or metabolism of the cell if the power required to maintain the temperature of the sample is less than the measured power of a reference sample that does not contain the agent.

40. The method of claim 39, further comprising digitally recording the change in thermal energy of the reference sample.

41. The method of claim 39, wherein the liquid sample is culture medium.

42. The method of claim 39, wherein the cell is isolated from a patient sample.

43. The method of claim 39, wherein the cell is isolated from a food source.

44. The method of claim 39, wherein the cell is isolated from feedstock sample.

45. The method of claim 39, wherein the liquid sample comprises a density of from about 10⁵ to about 10⁷ of the cell per mL of liquid sample.

46. The method of claim 39, wherein said measurement is performed by Isothermal Titrative Calorimetry.

47. The method of claim 39, wherein determining that the agent affects the growth or metabolism of the cell is performed by digital comparison.

48. The method of claim 39, wherein the sample containing the agent or the reference sample comprises a substantially homogenous population of cells.

49. The method of claim 48, wherein the substantially homogenous population comprises a clonal population.

50. The method of claim 39, wherein the sample containing the agent or the reference sample comprises a substantially heterogeneous population of cells.

51. The method of claim 39, further comprising performing multivariate analysis of the energy required to maintain the samples at a substantially constant temperature.

52. The method of claim 39, wherein the measurement is repeated with the same liquid sample.

53. The method of claim 39, wherein the measured energy is stored digitally.

54. The method of claim 51, further comprising digitally storing the results of the multivariate analysis.

55. A method for treating a patient in need thereof, comprising:
a. performing the method of any of claims 39 to 54; and
b. administering to the patient the agent determined to affect the growth or metabolism of the cell.

56. A system for identifying a cell, the system comprising:
a processor; and
a computer-readable medium operably coupled to the processor, the computer-readable medium comprising instructions that, upon execution by the processor, perform operations comprising measuring the change in the thermal energy of a liquid sample containing the cell as compared to a reference sample and correlating the change in the thermal energy to identify the classification of the cell.

57. A system for determining if an agent affects the metabolism of a cell, the system comprising:
a processor; and
a computer-readable medium operably coupled to the processor, the computer-readable medium comprising instructions that, upon execution by the processor, perform operations comprising measuring the change in the thermal energy of a liquid sample containing the agent and the cell as compared to a reference sample and identifying the agent that alters the change in the thermal energy of the cells in the sample.

58. A system for determining if an agent alters the metabolism of a cell, the system comprising:
a processor; and
a computer-readable medium operably coupled to the processor, the computer-readable medium comprising instructions that, upon execution by the processor, perform operations comprising measuring the amount of energy required to maintain the temperature of a liquid sample substantially constant as compared to a reference sample and identifying the agent that alters the amount of energy required to maintain the temperature of the liquid sample substantially constant.
